(19) United States
(12) Patent Application Publication
(10) Pub. No.: US 2018/0353258 A1
(43) Pub. Date: Dec. 13, 2018

(54) ILLUMINATED RETRACTOR

(71) Applicant: OBP Medical Corporation, Lawrence, MA (US)

(72) Inventors: Matthew Traub, Andover, MA (US); Douglas McConnell, Redding, CA (US); Jeffrey Ralph Swift, Boca Grande, FL (US)

(21) Appl. No.: 16/006,287

(22) Filed: Jun. 12, 2018

Related U.S. Application Data
(60) Provisional application No. 62/518,746, filed on Jun. 13, 2017.
(60) Provisional application No. 62/529,534, filed on Jul. 7, 2017.

(51) Int. Cl. A61B 90/35; A61B 17/02; A61B 17/88

(52) U.S. Cl. CPC A61B 90/35; A61B 17/88; A61B 17/02

(57) ABSTRACT

An illuminated surgical retractor, comprising a metal blade having a top surface and a bottom surface, a metal handle extending from a proximal end of the blade, and a plastic illumination housing assembly comprising at least one light source, at least one battery and an activation device for energizing the light source, and the illumination assembly being permanently and non-releasably attached to the metal handle and/or blade.

### CROSS-REFERENCE TO RELATED APPLICATIONS

This application claims priority to provisional application No. 62/518,746 field on Jun. 13, 2017 and U.S. provisional application No. 62/529,534 filed on Jul. 7, 2017, both of which are incorporated herein by reference.

### INTRODUCTION

Existing technology for illumination during surgical/medical procedures includes overhead illumination. This illumination comes from either overhead lighting or head mounted fiber optic systems. Traditional overhead lighting systems face numerous limitations. A direct exposure of the field from the overhead source is required. Changes in patient or surgeon positioning may interfere with the light source. Frequent adjustments provide an inconvenience for the surgeon and disrupt the surgical flow. Overhead lighting is frequently inadequate for surgery in deeper cavities where more intense focused illumination may be required.

In addition, the alignment of the surgeon's head frequently interferes with the remote illumination and prevents light from reaching the field. Head mounted fiber optic systems are used frequently for more limited surgical exposures. However, these devices have numerous limitations. First, the surgeon is tethered by the light cord attached to the headset, limiting the mobility in the operating room. Second, the devices are associated with head and neck fatigue with frequent or prolonged use. Third, the devices require the surgeon to maintain a steady head and neck position to provide a constant and steady illumination of the field.

Fourth, the use of remote light sources and fiber bundles introduces tremendous inefficiencies into the system. A typical ten-foot long cable will lose illumination by approximately 10% per foot of cable for a 300-watt light source, which results in much lower illumination than desired.

Fifth, a head lamp's illumination is not collinear with the doctor's eyes, and may cast shadows in the field of view when illuminating surgical cavities. Sixth, halogen bulbs get very hot and often burn the skin surrounding the surgical pocket the surgeon is working in.

Other existing technology for illumination during surgical/medical procedures includes lighted surgical retractors. These retractors include integral or attached light sources which project light locally down the retractor blade. Existing lighted surgical retractors overcome the problems with overhead illumination, but still suffer from several shortcomings. These retractors can generally be classified into two categories.

The first category includes those with detachable light sources. This category allows the retractor to be re-used and therefore the retractor must be sterilized prior to re-use. Characteristics of most light sources are not compatible with many sterilization procedures. For example, it is uncommon for batteries to carry out high temperature sterilization. It is also difficult to completely remove organic material from light source assemblies.

To overcome these difficulties, lighted surgical retractors with detachable light sources were created. These light sources are releasably attached to the retractor via tape or other adhesive or clip-on mechanism. This class of lighted surgical retractors requires assembly prior to use and disassembly, cleaning, and sterilization after use. Such assembly, disassembly, cleaning, and sterilization represent significant time, cost, and inefficiency for the user.

The second category of lighted surgical retractors consists of surgical retractors with light sources that are integrated into the retractor and are not removable. These lighted surgical retractors contain a power source in the retractor handle, an illumination device built into, or permanently attached to the blade, and some form of optical or electrical coupling between the power source and the illumination device. The power source can be batteries or a device that will plug into the wall. It could also be an optical power source that generates optical energy instead of electrical energy. The illumination device is either one or more light emitting diodes, filament light bulbs, a fiber optic cable, or an optical waveguide. The form of coupling is either wiring for an electrical connection, or a fiber optic cable or optical waveguide for optical coupling.

This second category of lighted surgical retractors reduces the problem of assembly and disassembly from which the first category of surgical retractors suffers. But this second class of retractors still suffers from difficulty in cleaning and sterilization.

Moreover, in order to be sterilizable (i.e., to withstand the thermal trauma of high pressure steam sterilization), surgical retractors have been generally made of stainless steel. If they had any attached lighting system this required hand disassembly after use, hand cleaning and then repackaging for gas sterilization of the lighting apparatus. This device then required reassembly on the surgical table prior to use.

Also, the known techniques involved in integrating light source components into the handle and blade are generally costly. Recent evidence is emerging that procedures for cleaning and sterilization are often flawed in practice, resulting in possible cross contamination of patients. These deficiencies have prevented a widespread adoption of this second category of lighted orthopedic retractors.

### SUMMARY OF THE INVENTION

Certain embodiments described herein represent a new class of lighted orthopedic retractors that does not suffer from these known deficiencies. These embodiments completely eliminate the risk of cross contamination by ensuring that each retractor can be only used once. These embodiments eliminate the costly electrical or optical interconnect systems required by previous orthopedic retractors. These embodiments also eliminate the requirement of assembly, disassembly, cleaning, and re-sterilization by the end user.

In one or more exemplary embodiments, an illuminated orthopedic retractor includes a metal blade, a metal handle, and an attached plastic housing comprising an illumination assembly. The illumination assembly includes at least one light source, at least one battery and an activation device for energizing the light source.

In one or more embodiments, the chemical capacity of the battery (or batteries) is sufficient for only a single use and the illuminated surgical retractor is discarded after the single use. The intrinsic low cost of these embodiments makes the illuminated retractor economically attractive, and eliminates the inefficiency and expense of cleaning and re-sterilization.

One exemplary aspect comprises an illuminated orthopedic retractor, comprising: a metal blade; a metal handle extending from a proximal end of the blade; and a plastic illumination assembly comprising at least one light source, at least one battery and an activation device for energizing the light source, with the illumination assembly being permanently and non-releasably attached to the metal handle and/or blade.

In another exemplary embodiment, the invention is directed to an illuminated surgical retractor comprising a retractor frame including a blade and a handle extending from a proximal end of the blade, and an illumination assembly comprising at least one light source, at least one battery, an activation device for energizing the light source, and a housing sleeve attached to the retractor frame and enclosing the at least one battery. In such embodiment, the housing sleeve is fitted around the retractor frame and encloses a portion of a length of the retractor frame. The housing sleeve may be formed from plastic and/or nylon, and may be fitted around the retractor frame by over-molding or shrink wrapping, or may be attached to the retractor frame by encapsulating the housing sleeve in epoxy material and hardening the epoxy material.

In another embodiment, the illuminated retractor comprises a blade having a top and a bottom surface, a handle extending from a proximal end of the blade, and an illumination assembly comprising at least one light source, at least one battery and an activation device, wherein the at least one light source is attached to a rotatable base portion rotatable relative to the retractor.

In certain exemplary embodiments, the illuminated surgical retractor includes a blade, a handle and an illumination assembly which is slidably mounted on a track attached to the retractor. In some exemplary embodiments, the illuminated surgical retractor includes a blade, a handle and an illumination assembly which includes a malleable neck attached to the light source for adjusting the angle of the light source relative to the retractor. In some exemplary embodiments, the illuminated surgical retractor comprises a blade, a handle and an illumination assembly which includes a vertical adjustment mechanism for adjusting a vertical angle of the light source relative to the retractor. The vertical adjustment mechanism may be in the form of an expandable support that expands and contracts or may use a ratchetable support that engages with the light source at one or more predetermined positions for adjusting the angle of the light source. Further features and advantages will be apparent to those skilled in the art after reviewing the drawings and detailed description provided herein.

### BRIEF DESCRIPTION OF THE DRAWINGS

FIG. 1 provides two views of a prior art steel orthopedic retractor.

FIGS. 2A-2C provide three views of an orthopedic retractor of an exemplary embodiment.

FIG. 3 depicts exemplary usage of an exemplary embodiment.

FIG. 4 depicts a top view of a movable light source comprising a malleable neck.

FIG. 5 depicts a side view of a movable light source comprising a malleable neck.

FIG. 6 depicts a side view of a retractor having a movable light source comprising a malleable neck, with a base of the light source mounted on a track attached to the retractor.

FIG. 7 depicts a top view of a retractor having a movable light source comprising a malleable neck, with a base of the light source mounted on a track attached to the retractor.

FIG. 8 depicts a top view of a movable light source comprising a rotatable base.

FIG. 9 depicts a side view of a movable light source comprising a rotatable base.

FIG. 10 depicts a side view of a movable light source comprising an expandable support.

FIG. 11 depicts a side view of a movable light source comprising a ratchetable support.

FIG. 12 depicts a wing track configuration and an inverse wing track configuration.

### DETAILED DESCRIPTION OF SELECT EXEMPLARY EMBODIMENTS

Drawings are used herein to describe select exemplary embodiments. For the sake of clear illustration, many practical details are explained together in the description below. However, the practical details described herein should not be used to limit the claim scope unless explicitly described in the claims. In other words, in some embodiments, certain details are not essential. Moreover, for the sake of drawing simplification, some customary structures and elements in the drawings are schematically shown in a simplified way.

Unless otherwise defined, all terms (including technical and scientific terms) used herein have the same meaning as commonly understood by one of ordinary skill in the art. It should be further understood that terms, such as those defined in commonly used dictionaries, should be interpreted as having a meaning that is consistent with their meaning in the context of the relevant art and the present description, and should not be interpreted in an idealized or overly formal sense unless expressly so defined herein.

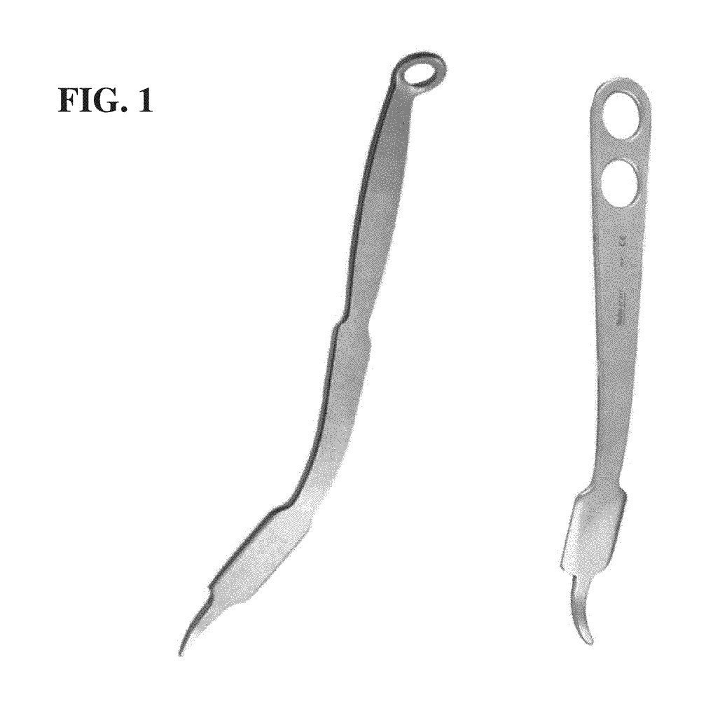

FIG. 1 provides two views of a prior art orthopedic retractor. Typically such retractors are constructed of a metal such as stainless steel.

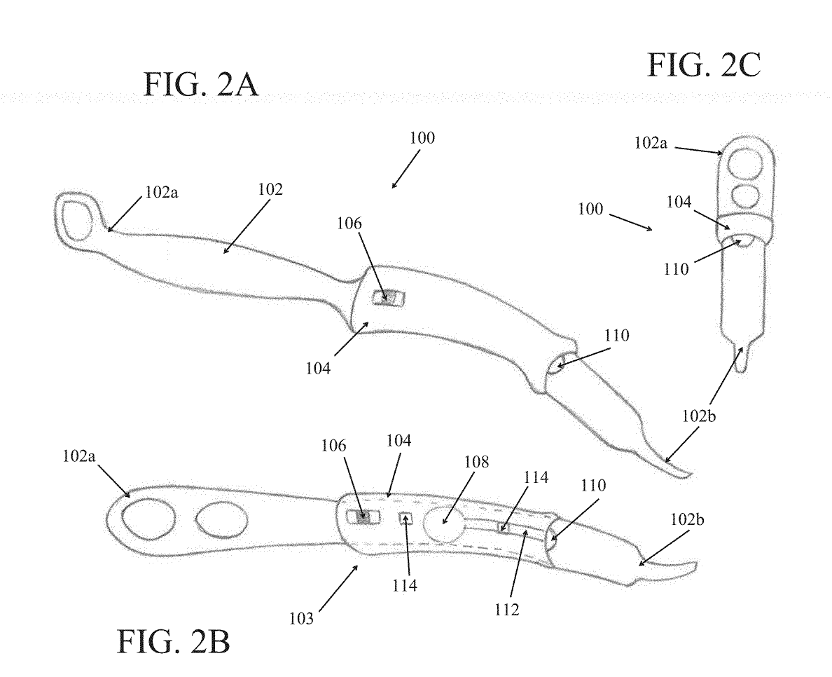

FIGS. 2A-2C provide three views of an orthopedic retractor 100 of an exemplary embodiment. The retractor 100 includes a retractor frame 102, which has a handle portion 102a at one end and a blade portion 102b at the other end. In the present illustrative embodiment, the handle portion 102a and the blade portion 102b of the frame are integrally formed. However, in other embodiments, the handle portion 102a and the blade portion 102b may be formed separately and attached to one another using any suitable mechanism and the orientation of the handle may be adjustable relative to the orientation of the blade portion. The retractor frame 102 may be formed from metal, such as stainless steel or titanium, or from one or more polymers, such as a glass fiber-reinforced polymer, a glass fiber-reinforced polyarylamide, glass fiber-reinforced acrylonitrile butadiene styrene (ABS) or any other suitable plastic or polymer having sufficient strength.

In FIG. 2A, the shaded section on the retractor is the plastic housing 104. An on/off switch 106 (alternatively, an on-only switch such as a pull tab), one or more batteries 108, at least one light source 110 (LED, in this embodiment), and leads 112 connecting the switch 106, one or more batteries 108, and the at least one light source 110, are integrated within the housing 104 configured to fit as a sleeve onto the retractor frame 102 of the retractor 100. Those components are depicted in further detail in FIG. 2B, and FIG. 2C shows the retractor 100 and the housing 104 from an end-view perspective. Although the exemplary embodiment shown in FIGS. 2A-2C has one light source 110, in other embodiments, multiple light sources 110 are used to provide additional and/or more targeted and selective illumination. The orientation, e.g., angle, of the light sources 110 may be adjustable. The switch 106 may be configured to control the turning on and off of the individual light sources 110 so as to provide selective illumination. In addition, the switch 106 may be configured to adjust the brightness of individual one or more light sources 110.

In an exemplary embodiment, one or more holes or slots 114 in the retractor frame 102 assist in aligning the housing 104 with the frame 102. In certain configurations, these holes or slots 114 engage with corresponding protrusions formed on the inner surface of the housing 104 in order to align the housing 104 with the frame 102.

In an exemplary embodiment, the housing 104 is attached to the metal or polymer retractor frame 102 during the manufacturing process. In another embodiment, the housing 104 may be attached to an existing (e.g., prior art) metal (e.g., stainless steel) frame 102. In the latter embodiment, one or more slots may be created in the metal frame 102 (using methods that will be apparent to those skilled in the art, such as a punch or drill) in order to improve alignment of the housing 104. In yet other embodiments, the retractor frame and the housing may be formed integrally so that the entire retractor, including the frame and the housing, are formed as a single piece.

Various types of plastic, polymer or similar materials (e.g., nylon, silicone) may be used for the housing 104, and various methods of attaching the housing 104 to the retractor frame 102 may be used. For example, a plastic housing assembly 103 that (at least partially) includes the housing 104 and contains the batteries 108, the switch 106, and the at least one light source 110 may be permanently attached with shrink wrap to the retractor frame 102. In another embodiment, the housing assembly 103 is over-molded in plastic, and in another embodiment the housing assembly is encapsulate in an epoxy-type substance that hardens.

While the housing assembly 103 is designed to provide sufficient illumination for only a single use, an embodiment comprises an on/off switch 106 so that power may be conserved during a lengthy surgical operation. However, the housing 104, in certain embodiments, is designed and configured so as to discourage use on multiple patients.

In one or more embodiments, the chemical capacity of the battery (or batteries) (in one embodiment, approximately 150 mAh) is sufficient for only a single use and the illuminated surgical retractor is discarded after the single use. Most low voltage LEDs are rated at 20 mA, but can be over-driven to 50 mA or more for additional brightness and shorter life. An exemplary embodiment over-drives the LED(s) to maximize brightness and to get a few (1-2) hours of operation from 150 mAh batteries.

In another embodiment, the batteries 106 are connected to the one or more light sources 110, e.g., LED(s), through a power conversion circuit (not shown) that has the capability of turning the LED(s) on or off all together, or turn the LED(s) on or off in a sequence that allows for partial lighting when multiple LED(s) are used, and/or a dimming capability that allows a user to adjust the light intensity. Such power conversion circuit may be included in a switch 106 that has a slide or rotating control to control the brightness and a pushbutton control for on/off or sequencing operation. In another embodiment the slide or rotating control is combined with the pushbutton action to control both features through one device.

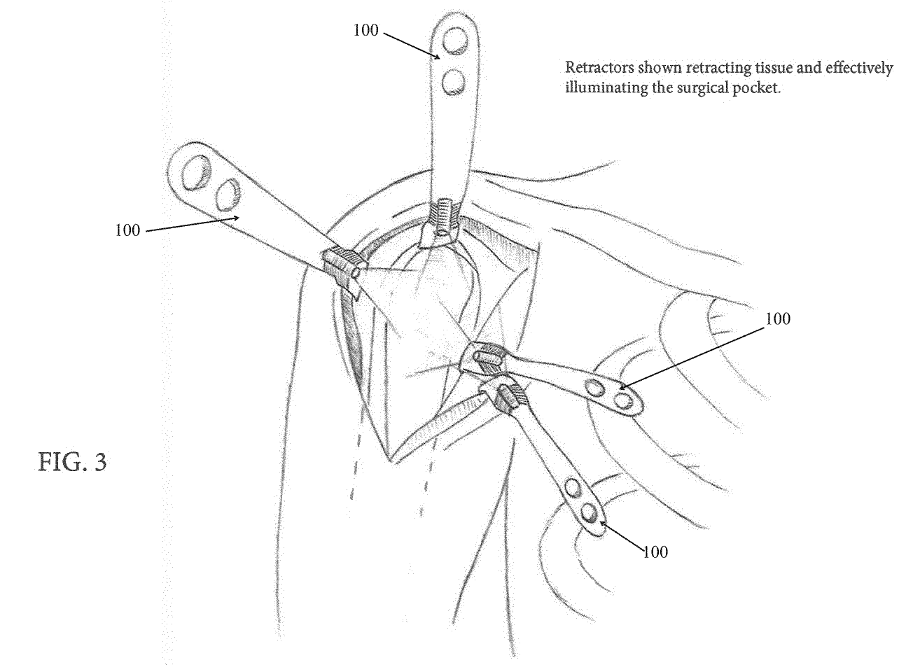

FIG. 3 depicts usage of an exemplary embodiment of the retractors 100 of FIGS. 2A-2C. Retractors 100 are shown retracting tissue and effectively illuminating a surgical pocket.

As mentioned above, while one or more exemplary embodiments comprise a housing for a metal orthopedic retractor, those skilled in the art will understand that other embodiments may be suitable for use with other types of (metal and/or non-metal) retractors and surgical instruments. Moreover, while most metal retractors are constructed of stainless steel, it should be understood that the embodiments described herein will work with other types of metal.

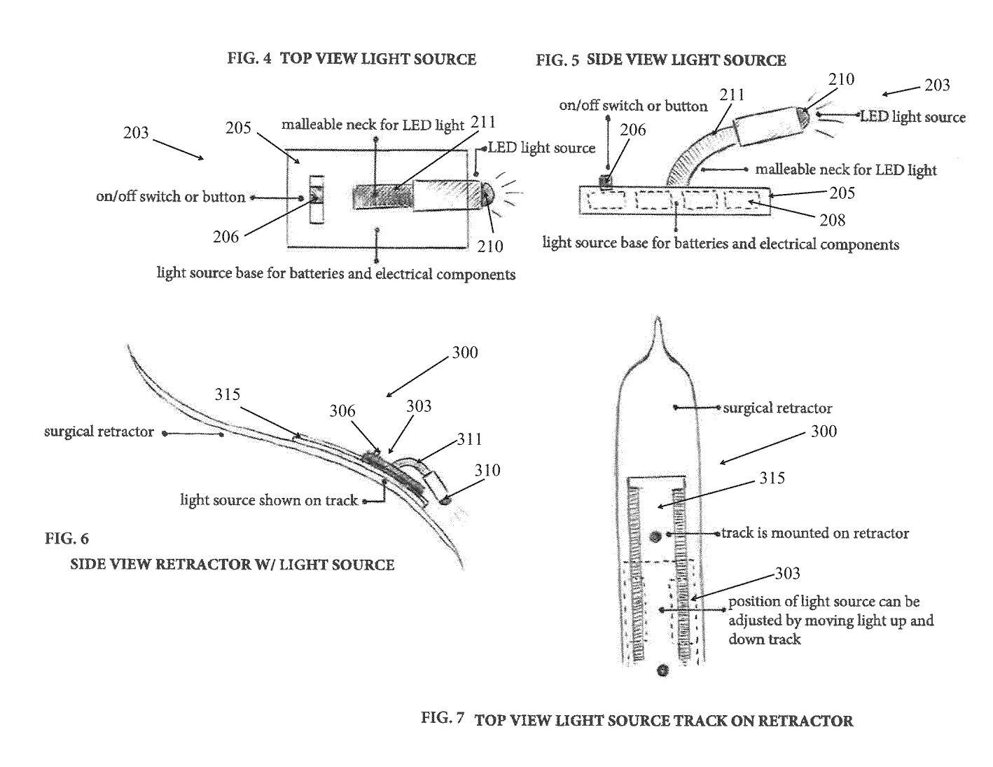

Types of retractors for which at least one embodiment described herein is suitable include, but are not limited to, the following: Hohmann Retractor; Acetabular Hip Retractor; Cobra Retractor; Superior Capsular Retractor; Right Angle Posterior Capsular Retractor; Posterior-Inferior Retractor; Femoral Neck Elevator; Flanged Femoral Elevator; Offset Handle Hip Retractor; O'Reilly Direct Access Anterior Broaching Retractor; Hibbs Retractor; Soft Tissue Retractor; Darrach Retractor; Ragnell Retractor; Deltoid Retractor; Humeral Head Retractor; Knee Retractor; Nerve Retractor; Cloward Style Retractor; and Nerve Root Retractor. These types of retractors may be used with the housing assembly 103 of FIGS. 2A-2C described above as well as with assemblies described below and shown in FIGS. 4-11.

Other exemplary embodiments comprise one or more movable light sources that may be mounted on any type of retractor—i.e., is not limited to metal or orthopedic retractors. Non-limiting examples of such embodiments are depicted in FIGS. 4-11.

FIGS. 4-5 depict top and side views, respectively, of an illumination assembly 203 that includes at least one movable light source 210 comprising a malleable neck 211 mounted on a base 205. Exemplary materials for the malleable neck 211 include copper, tin, steel and lead tubing, and generally include metal and metal allow that retains its shape once bent. Other metals or combinations of metals and plastics may be used for the malleable neck 211. For example, malleable metallic wiring or tubing may be used for the malleable neck 211.

As shown in FIG. 5, the base 205 houses one or more batteries 206 and includes an ON/OFF or an ON only switch 206 on one of its surfaces. In FIG. 5, the switch 206 extends through an upper surface of the base 205 and the malleable neck 211 with the light source 210 is also coupled to the upper surface of the base 205. The lower surface of the base 205 is adapted for coupling to the retractor directly or via a track, as described below. In addition, electrical components, such as leads electrically connecting the light source 210, the batteries 208 and the switch 206 are housed in the base 205 and may pass through the malleable neck 211. In certain embodiments, the malleable neck 211 may include a non-conductive cover thereon, such as a plastic, silicone or polymer cover that is flexible, elastic and/or bendable, and the cover encloses the leads to the light source 210.

Similar to the assembly in FIGS. 2A-2C, in certain embodiments, the illumination assembly 203 may include multiple light sources 210. In such embodiments, each light source may be attached to the base 205 at a different location along the base using a respective malleable neck 211. In some embodiments, one malleable neck 211 may include multiple branches, each of which is connected to a respective light source. Other configurations of the light sources are also possible.

As discussed above, in some embodiments, the batteries 208 may be connected to the one or more light sources through a power conversion circuit (not shown) that can turn the light source(s) on or off together or in a sequence, or individually, to allow for selective and partial lighting when multiple light sources are used. The power conversion circuit may also have a dimming capability for adjusting the light intensity. In such embodiments, the power conversion circuit may be housed in the base 205 and the switch 206 may include a slide or rotating control and/or may be combined with a pushbutton to allow for multiple adjustments and functions in a single switch device.

FIGS. 6-7 depict side and top views, respectively, of a retractor 300 having a movable illumination assembly 303 comprising at least one light source 310 connected to a base 305 via a malleable neck 311, as described above with respect to FIGS. 4 and 5, and with the base 305 of the illumination assembly 303 mounted on a track 315 attached to the retractor 300. The configuration of the retractor 300 may be as described above with respect to FIGS. 2A-C or may be any other type of surgical retractor. Exemplary materials for the track 315 include metals, such as steel (especially stainless steel) or aluminum, that may be riveted or welded onto the retractor, or plastics and/or polymers, or any other suitable materials. The lower surface of the base 305 includes one or more mounting portions (not shown) configured to engage with the track 315. The mounting portions may be integrally formed with the base 305 or may be attached to the base 305 using any suitable means, e.g., adhesive, connectors, etc. The track 315 may include a plurality of locking positions for locking the base 305 into place relative to the track 315.

The malleable neck 311 allows a user to angle the light source 310 as desired, without being forced to move or change the angle of the retractor 300. Mounting the base 305 to a track 311 attached to a retractor allows the user to change the relative position of the light source 310 to the retractor 300—for example, to move the light source 310 closer to or further away from the work area or other area of interest. Of course, a fixed light source (i.e., without a malleable neck) may also be mounted to a track attached to a retractor. Exemplary track configurations include: a “wing” shape, an inverted “wing” shape (see FIG. 12), and any other shape attached to a longitudinal blade that is perpendicular to the retractor surface.

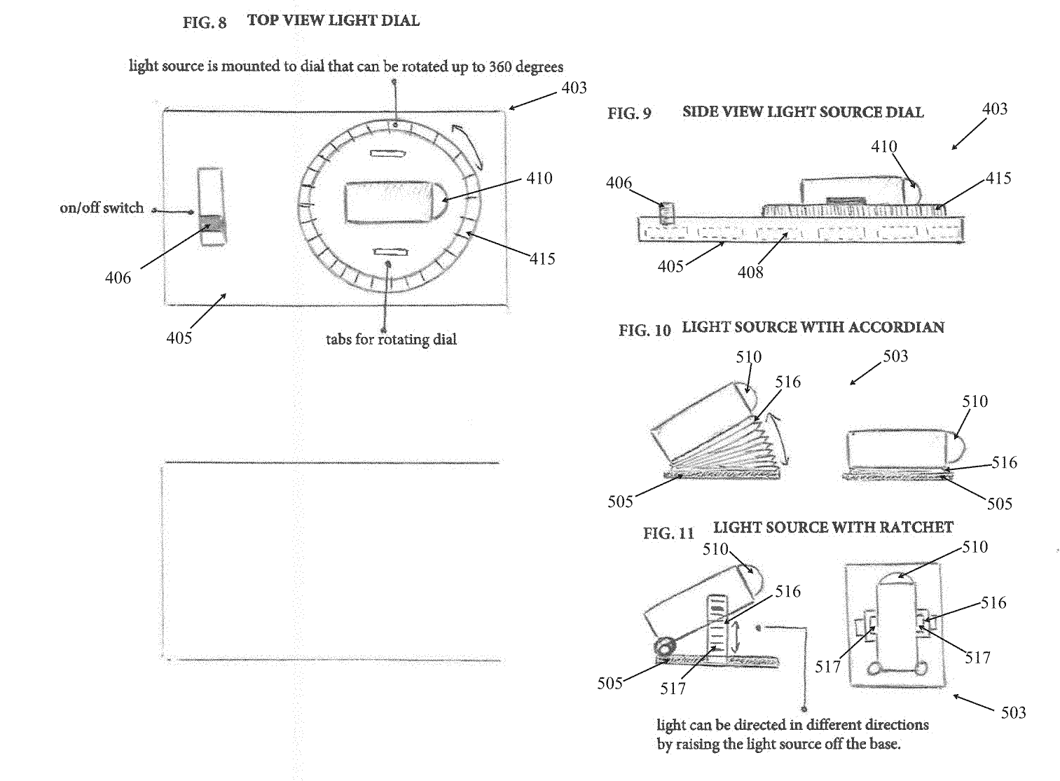

FIGS. 8-9 depict top and side views, respectively, of an illumination assembly 403 with at least one movable light source 410, the assembly 403 comprising a rotatable base portion 415. As in the previous embodiments, the illumination assembly includes a base 405 that houses the batteries 408, the electrical components and the leads between the batteries 408, the at least one light source 410 and the switch 406. The illumination assembly 403 shown in FIGS. 8-9 includes one light source 410, but in other embodiments, multiple light sources 410 may be used, with the multiple light sources 410 provided on the same rotatable base portion 415 or on individual rotatable base portions. As in the embodiments described above, a power conversion circuit may be used for controlling the turning on and off of the light source(s) and the brightness of the light source(s).

In the depicted examples, the rotatable base portion 415 is shown as a dial, but those skilled in the art will understand that other rotatable base portions are within the scope of the invention. The rotatable base portion 415 may be formed from plastic, or other materials, including metals, polymers, resins, etc. The rotatable base portion 415 is rotatably coupled to the base 405, and in the illustrative embodiment, the rotatable base portion 415 is rotatably coupled to the upper surface of the base 405. The rotatable base portion 415 supports thereon one or more light sources 410, which move together with the rotatable base portion 415. The rotatable base portion 415 allows a user to rotate the light source(s) 410 with respect to the base 405 and the retractor on which the illumination assembly is provided. In the embodiment of FIG. 8, the rotatable base portion 415 can be rotated up to 360 degrees, but in other embodiments, the rotation range of the rotatable base portion 415 may be limited.

In certain embodiments, the rotatable base portion 415 includes tabs or projections for easy rotation thereof. In addition, detent marks around the periphery of the rotatable base portion are used to indicate different positions. In an embodiment, the rotatable base portion 415 may also be raised in a vertical angular manner (away from the retractor) in order to increase the angle of the light relative to the surface of the retractor.

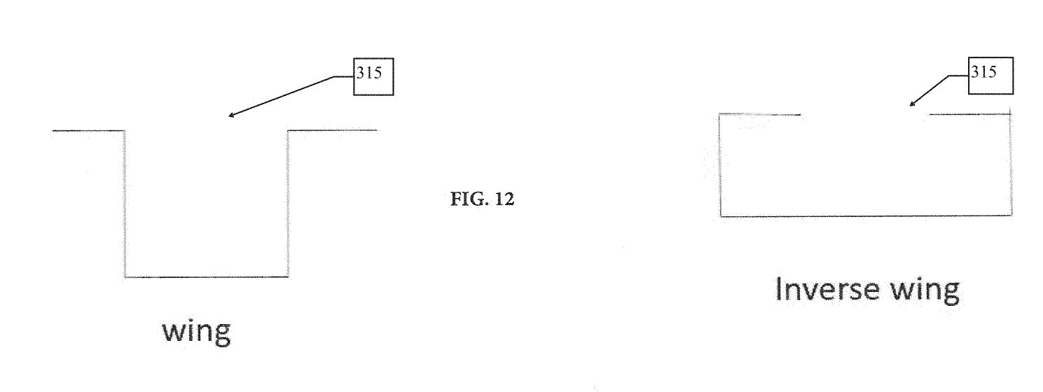

Alternatively, the light source 410, rather than the rotatable base portion 415, may be vertically adjustable relative to the rotatable base portion 415, as shown in FIGS. 10 and 11 and described in more detail below. Although FIGS. 8-9 show the rotatable base portion 415 being mounted on the base 405, in other embodiments, the rotatable base portion 415 with the light source(s) 410 may be mounted directly on the retractor and the other components, i.e., batteries, switch and electrical components, may be provided in the handle of the retractor or using a housing attached to the blade or the handle of the retractor.

FIGS. 10 and 11 show an illumination assembly 503 with at least one light source 510 and a vertical adjustment mechanism 516 for vertically adjusting the angle of the light source 510 relative to the retractor or relative to the base 505 of the illumination assembly 503. As in the previous embodiments, the base 505 houses therein the batteries, the leads and other electrical components, and may include a switch for turning the light source(s) on and off. In other embodiments, the light source(s) 510 and the vertical adjustment mechanism 516 can be attached directly to the retractor, without using a separate base, and the batteries, the leads, other electrical components and the switch may be provided in the retractor, such as in the handle of the retractor.

FIG. 10 depicts a side view of the movable light source 510 and the vertical adjustment mechanism 516 comprising an expandable (“accordion”) support. The expandable support allows a user to change the angle of the light source with respect to the retractor and the base 505. The expandable support is configured so as to retain or lock the position of the light source 510 after adjustment.

FIG. 11 depicts a side view of the movable light source 510 and the vertical adjustment mechanism 516 comprising a ratchetable support. In FIG. 11, one end of the light source 510 is hinged to the base 505, while another end can be raised or lowered relative to the base 505 and can be locked relative to the ratchetable support 516 in one or more predetermined positions. The ratchetable support allows a user to change the angle of the light source with respect to the retractor and the base 505. Exemplary materials for the ratchetable support include plastic, polymers, resins, metals and combinations thereof. The ratchetable support may include one ratchet portion 517 extending from the base 505 and adjustably coupled with a side of the light source 510, or may include two ratchet portions 517 that extend one each side of the light source 510. The light source 510 includes a locking portion provided on its side for engagement with one or more predetermined positions along the ratchet portion 517. Other configurations of the ratchetable support may be used.

Those skilled in the art will understand that the above-described embodiments may be used either separately or in combination. For example, the rotatable base portion, the expandable support, and the ratchetable support may be fixed to a base, which is attached to a retractor, may be fixed directly to the retractor or may be fixed to a base which is attached to a track that is attached to a retractor. Moreover, the expandable support and the ratchetable support may be used on a rotatable base portion.

Although the invention has been described in considerable detail herein with reference to certain embodiments thereof, other embodiments are possible. Therefore, the spirit and scope of the appended claims should not be limited to any non-claimed details of the embodiments contained herein. It will be apparent to a person having ordinary skill in the art that various modifications and variations can be made to the embodiments described herein without departing from the scope or spirit of the claimed invention.

1. An illuminated surgical retractor, comprising:
a metal blade having a top surface and a bottom surface;
a metal handle extending from a proximal end of the blade; and
a plastic illumination housing assembly comprising at least one light source, at least one battery and an activation device for energizing the light source, and the illumination assembly being permanently and non-releasably attached to the metal handle and/or blade.

2. An illuminated surgical retractor comprising:
a retractor frame including a blade and a handle extending from a proximal end of the blade;
an illumination assembly comprising at least one light source, at least one battery, an activation device for energizing the light source, and a housing sleeve attached to the retractor frame and enclosing the at least one battery,
wherein the housing sleeve is fitted around the retractor frame and encloses a portion of a length of the retractor frame.

3. The illuminated surgical retractor of claim 2, wherein the housing sleeve is formed from plastic.

4. The illuminated surgical retractor of claim 2, wherein the housing sleeve is formed from nylon.

5. The illuminated surgical retractor of claim 2, wherein the housing sleeve is fitted around the retractor frame by one or more of over-molding and shrink wrapping.

6. The illuminated surgical retractor of claim 2, wherein the housing sleeve is attached to the retractor frame by encapsulating the housing sleeve in epoxy material and hardening the epoxy material.

7. The illuminated surgical retractor of claim 2, wherein the retractor frame includes one or more slots for aligning the housing sleeve in a predetermined position relative to the retractor frame.

8. The illuminated surgical retractor of claim 2, wherein the retractor frame is a metal retractor frame and the housing sleeve is plastic.

9. The illuminated surgical retractor of claim 2, wherein the retractor frame is an orthopedic retractor.

10. An illuminated surgical retractor, comprising:
a blade having a top surface and a bottom surface;
a handle extending from a proximal end of the blade; and
an illumination assembly comprising at least one light source, at least one battery and an activation device for energizing the light source,
wherein the at least one light source is attached to a rotatable base portion rotatable relative to the retractor.

11. The illuminated surgical retractor of claim 10, wherein the rotatable base portion comprises a plastic dial.

12. The illuminated surgical retractor of claim 11, wherein the plastic dial includes one or more tabs formed on its surface.

13. The illuminated surgical retractor of claim 10, wherein:
the illumination assembly further comprises a base attached to the retractor and housing the at least one battery, and
the rotatable base portion is rotatably mounted on the base.

14. The illuminated surgical retractor of claim 10, wherein the rotatable base portion is rotatably mounted on the blade.

15. An illuminated surgical retractor, comprising:
a blade having a top surface and a bottom surface;
a handle extending from a proximal end of the blade; and
an illumination assembly comprising at least one light source, at least one battery, an activation device for energizing the light source,
wherein the illumination assembly is slidably mounted on a track attached to the retractor.

16. The illuminated surgical retractor of claim 15, wherein:
the illumination assembly comprises a base housing the at least one battery,
the at least one light source is mounted on the base, and
the base is slidably mounted on the track.

17. The illuminated surgical retractor of claim 16, wherein the activation device is provided in the base.

18. The illuminated surgical retractor of claim 15, wherein the track is attached along at least a portion of a length of the blade.

19. The illuminated surgical retractor of claim 16, wherein the at least one light source is mounted on the base via a malleable neck.

20. An illuminated surgical retractor, comprising:
a blade having a top surface and a bottom surface;
a handle extending from a proximal end of the blade; and
an illumination housing assembly comprising at least one light source, at least one battery and an activation device for energizing the light source, and the illumination assembly further comprising a malleable neck attached to the light source and for adjusting the angle of the light source relative to the retractor.

21. An illuminated surgical retractor, comprising:
a blade having a top surface and a bottom surface;
a handle extending from a proximal end of the blade; and
an illumination housing assembly comprising at least one light source, at least one battery and an activation device for energizing the light source, and the illumination assembly further comprising a vertical adjustment mechanism for adjusting a vertical angle of the light source relative to the retractor.
expandable support for the light source, the expandable support being adapted.

22. The illuminated surgical retractor of claim 21, wherein the vertical adjustment mechanism comprises an expandable support configured to expand and contract for adjusting the angle of the light source relative to the retractor.

23. The illuminated surgical retractor of claim 21, wherein the vertical adjustment mechanism comprises a ratchetable support configured to engage with the light source at one or more predetermined positions along the ratchetable support for adjusting the angle of the light source relative to the retractor.

24. The illuminated surgical retractor of claim 21, wherein the illumination housing assembly is attached to a rotatable base portion configured to rotate relative to the retractor.

25. The illuminated surgical retractor of claim 21, wherein the illumination housing assembly is mounted on a track attached to the retractor.
